(19) United States
(12) Patent Application Publication
(10) Pub. No.: US 2011/0274593 A1
(43) Pub. Date: Nov. 10, 2011

(54) PROCESSES FOR PREPARING A POLYMERIC INDICATOR FILM

(75) Inventors: Joel R. Gorski, Perrysburg, OH (US); Jon Booher, Advance, NC (US)

(73) Assignee: Indicator Systems International, Inc.

(21) Appl. No.: 13/010,725

(22) Filed: Jan. 20, 2011

Related U.S. Application Data
(60) Provisional application No. 61/297,234, filed on Jan. 21, 2010.

(51) Int. Cl. G01N 33/52; C09J 5/02

(52) U.S. Cl. 422/400; 156/60

(57) ABSTRACT

Disclosed herein are processes for preparing a polymeric indicator film which can be used to determine the presence of bacterial growth in fluids and substances which contact the film. In particular, in the disclosed processes, a pH indicator is incorporated into the polymeric film. The incorporated pH indicator in the film remains transparent under neutral conditions, whereas under acidic conditions, the pH indicator in the film imparts a distinguishable color to the film.

### CROSS-REFERENCE TO RELATED APPLICATIONS

This application claims the benefit under 35 U.S.C. §119(e) of U.S. Provisional Application No. 61/297,234, filed Jan. 21, 2010, which is hereby incorporated by reference in its entirety.

### BACKGROUND

1. Field of the Invention

This invention is directed to processes for preparing a polymeric indicator film which can be used to determine changes in pH in fluids in contact therewith. In particular, in the disclosed processes, a pH indicator is incorporated into the polymeric film. In some embodiments, the incorporated pH indicator maintains the film's transparency under neutral conditions, whereas under acidic conditions, the pH indicator in the film imparts a distinguishable color to the film.

As growth of microorganisms, such as bacteria, generates byproducts which when combined with water, alters the pH of the water from neutral to acidic, the pH indicators in the film are useful as they will impart color to the film evidencing bacterial growth on the material in contact with the film.

2. State of the Art

Heretofore, the art has disclosed a variety of polymeric films having embedded therein a pH indicator so as to provide for evidence of bacterial growth. Typically, the polymeric film has at least two layers. An inner layer which is hydrophilic and allows the transport of hydronium (H⁺) ions and an outer layer which is hydrophobic and imparts a barrier to water transport across the polymeric film. The inner layer is allowed to contact aqueous fluids such as those generated from food or body fluids. Bacterial growth is known to generate a variety of by-products such as carbon dioxide, hydrogen sulfide, etc. which when contacted with an aqueous solution at neutral pH will increase the acidity of the solution.

In one embodiment of the prior art, cavities are generated in the inner or hydrophilic layer of the polymeric film such that the pH indicator could be embedded in those cavities. See, e.g., Horan, et al., U.S. Pat. No. 5,753,285 (Horan). In another embodiment, pH indicators are covalently bound to at least a portion of a monomeric component used to prepare the hydrophilic layer of the polymeric film. See, e.g., Booher, et. al., WO 2009/061831 (Booher).

Nevertheless, both Horan and Booher required significant manufacturing challenges and processes for preparation of such films are quite involved. For example, it is quite difficult to entrap a sufficiently high density of pH indicator into the hydrophilic layer so that an intense color change can be visualized.

Accordingly, a process for the preparation of polymeric indicator films that permit an intense color change would be desirable.

### SUMMARY OF THE INVENTION

This invention is directed to processes for preparing a polymeric indicator film for detecting a change in pH. The processes provide for high densities of pH indicators such that intense color change is provided upon a change in pH.

Accordingly, in one of its process aspects, there is provided a process for preparing a polymeric indicator film comprising an inner hydrophilic, hydronium ion penetrating transparent layer and an outer hydrophobic, water impermeable transparent layer which process comprises:
a) selecting one or more hydrophilic, hydronium ion penetrating layers as the inner layer of the polymeric film wherein the inner layer has a first and second surface;
b) selecting one or more hydrophobic, water impermeable layers as the outer layer of the polymeric film wherein the outer layer has a first and second surface;
c) applying a pH indicator layer to at least a portion of one surface of the outer layer; and
d) bonding the inner and outer surfaces together such that the pH indicator layer is placed between the inner and outer layers.

In some embodiments, the process further comprises step c1) applying an adhesive layer to the one surface of the outer layer on which the pH indicator layer will be applied, which step is formed prior to step c).

In another aspect, this invention provides a polymeric indicator film comprising:
a) an inner layer comprising one or more hydrophilic, hydronium ion penetrating layers;
b) an outer layer comprising one or more hydrophobic, water impermeable layers;
c) a pH indicator layer placed between the inner and outer layer.

In some embodiments, the pH indicator is selected from the group consisting of xylenol blue (p-xylenolsulfonephthalein), bromocresol purple (5′,5″-dibromo-o-cresolsulfonephthalein), bromocresol green (tetrabromo-m-cresolsulfonephthalein), cresol red (o-cresolsulfonephthalein), phenolphthalein, bromothymol blue (3′,3″-dibromothymolsulfonephthalein), p-naphtholbenzein (4-[alpha-(4-hydroxy-1-naphthyl)benzylidene]-1(4H)-naphthalenone), neutral red (3-amino-7-dimethylamino-2-methylphenazine chloride), hexamethoxy red and heptamethoxy red, among others.

In some embodiments, the pH indicator is selected from the group consisting of hexamethoxy red and heptamethoxy red. These indicators provide a distinct advantage of providing an uncolored transparent polymeric film at neutral pH and an intense red color at acidic pH.

### BRIEF DESCRIPTION OF THE DRAWINGS

FIG. 1 illustrates one example of a polymeric indicator film prepared by the processes of this invention.

FIG. 2 illustrates a first example of the polymeric indicator film preparation process.

FIG. 3 illustrates a second example of the polymeric indicator film preparation process.

FIGS. 4-10 provide a side-by-side comparison evidencing the ability of the pH indicating films of this invention to readily determine the presence of bacterial growth on food stuffs.

### DETAILED DESCRIPTION OF THE INVENTION

As noted above, this invention is directed to processes for preparing a polymeric indicator film for detecting a change in pH. The films prepared by the processes of this invention can be used to detect the growth of microorganisms, such as bacteria in, for example, food or body fluids. However, prior to describing this invention in further detail, the following terms will first be defined.

### DEFINITIONS

As used herein, the following terms have the following meanings:

As used herein, certain terms may have the following defined meanings. As used in the specification and claims, the singular form “a,” “an” and “the” include singular and plural references unless the context clearly dictates otherwise.

The term “comprising” is intended to mean that the compounds and methods include the recited elements, but not excluding others. “Consisting essentially of” when used to define compositions and methods, shall mean excluding other elements of any essential significance to the compounds or method. “Consisting of” shall mean excluding more than trace elements of other ingredients for claimed compounds and substantial method steps. Embodiments defined by each of these transition terms are within the scope of this invention. Accordingly, it is intended that the processes and compositions can include additional steps and components (comprising) or alternatively include additional steps and compounds of no significance (consisting essentially of) or alternatively, intending only the stated methods steps or compounds (consisting of).

The term “polymeric indicator film” refers to a film having at least an inner hydrophilic, hydronium (H⁺) ion penetrating layer and an outer, water impermeable layer wherein sandwiched between the inner and the outer layer is a pH indicating layer comprising one or more pH indicators.

The term “neutral pH” refers to physiologically neutral pHs such as a pH of from about 5 to about 8 or 5 to 8.

The term “acidic” as used herein refers to an acidic pH range generally produced from by-products of bacterial growth. Such acidic pHs generally range from above 1 to about 5 and, preferably, a pH range of from 2 to 5.

The term “transparent layer” refers to a polymer layer which is sufficiently transparent to visible light that a viewer can readily see through the layer.

The term “hydrophilic, hydronium ion penetrating transparent layer” refers to a transparent polymer layer as defined above wherein the polymer is hydrophilic and permits protons (hydronium ions) to readily penetrate into the layer. Proton penetration can be determined by a number of measures but is most easily measured by the use of a pH indicator which detects the presence of a sufficient number of protons by a color change. Examples of hydrophilic, hydronium ion penetrating transparent layers include several which are commercially available materials having high moisture vapor transmission rate, such as polyether block amide copolymers (e.g., Pebax®). Preferred hydrophilic, hydronium ion penetrating polymer layers are those which qualify as food grade polymers.

The term “hydrophobic, water impermeable layer” refers to a transparent polymer layer as defined above wherein the polymer is hydrophobic and does not permit water or protons (hydronium ions) to readily penetrate into the layer. Especially preferred hydrophobic, water impermeable layers are those which qualify as food grade polymers.

The term “bonding” refers the formation of a single polymeric film by adhering two or more separate layers into a single film by conventional techniques.

The term “heptamethoxy red” refers to the chemical 2,4,6,2′,4′,2″,4″-heptamethoxytriphenylcarbinol having Formula (I) below. The term “hexamethoxy red” refers to the chemical 2,4,2′,4′,2″,4″-hexamethoxytriphenylcarbinol having Formula (II) below.

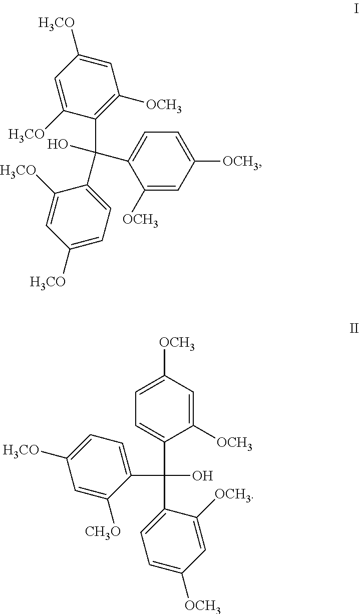

Heptamethoxy red exhibits a dynamic range between approximately pH 7 and pH 5, and hexamethoxy red exhibits a dynamic range between approximately pH 4.5 and pH 2.6. Both exhibit visually perceptible color change from colorless to colorful (reddish) when exposed to acid.

The term “food spoilage” refers to the growth of microorganisms, such as bacteria, on food.

The term “about” when used before a numerical value indicates that the value may vary within reasonable range, such as ±10%, ±5%, and ±1%.

### Processes

In one of its process aspects, there is provided a process for preparing a polymeric indicator film comprising an inner hydrophilic, hydronium ion penetrating transparent layer and an outer hydrophobic, water impermeable transparent layer which process comprises:
a) selecting one or more hydrophilic, hydronium ion penetrating layers as the inner layer of the polymeric film wherein the inner layer has a first and second surface;
b) selecting one or more hydrophobic, water impermeable layers as the outer layer of the polymeric film wherein the outer layer has a first and second surface;
c) applying a pH indicator layer to at least a portion of one surface of the outer layer; and
d) bonding the inner and outer surfaces together such that the pH indicator layer is placed between the inner and outer layers.

In some embodiments, the process further comprises step c1) applying an adhesive layer to the one surface of the outer layer on which the pH indicator layer will be applied, which step is performed prior to step c).

In some embodiments, there is provided a process for preparing a polymeric indicator film comprising an inner hydrophilic, hydronium ion penetrating transparent layer and an outer hydrophobic, water impermeable transparent layer which process comprises:
a) selecting one or more hydrophilic, hydronium ion penetrating layers as the inner layer of the polymeric film wherein the inner layer has a first and second surface;
b) selecting one or more hydrophobic, water impermeable layers as the outer layer of the polymeric film wherein the outer layer has a first and second surface;
c) applying an adhesive layer to one surface of the outer layer;
d) applying a coating of a pH indicator to at least a portion of the adhesive layer; and
e) bonding the inner and outer layers together such that the adhesive layer coated with the pH indicator is placed between the inner and outer layers, thereby forming the polymeric indicator film.

In some embodiments, the adhesive is partially dried before the coating of the pH indicator is applied. In some embodiments, the outer layer with the indicator applied thereto is dried before binding of the outer layer with the inner layer.

In one embodiment, the pH indicator is selected from the group consisting of xylenol blue (p-xylenolsulfonephthalein), bromocresol purple (5′,5″-dibromo-o-cresolsulfonephthalein), bromocresol green (tetrabromo-m-cresolsulfonephthalein), cresol red (o-cresolsulfonephthalein), phenolphthalein, bromothymol blue (3′,3″-dibromothymolsulfonephthalein), p-naphtholbenzein (4-[alpha-(4-hydroxy-1-naphthyl)benzylidene]-1(4H)-naphthalenone), neutral red (3-amino-7-dimethylamino-2-methylphenazine chloride), hexamethoxy red and heptamethoxy red, and combinations thereof.

In one embodiment, the pH indicator comprises hexamethoxy red. In one embodiment, the pH indicator comprises heptamethoxy red.

In some embodiments, the pH indicator layer comprises a sufficient amount of pH indicator moieties to provide visible color change in at least a portion of the polymeric pH indicator film upon contact with microorganism growth.

In some embodiments, the pH indicator layer has a thickness of from about 200 Angstroms (Å) to about 5 microns.

The adhesive layer may be any adhesive suitable for the application of bonding the pH indicator(s) to the outer layer so that the pH indicator(s) do not escape from the inner layer of the polymeric pH indicator film. Preferably the adhesive is non-toxic (more preferably food grade) when dried and has a neutral or slightly basic pH, for example a pH not significantly above the pKa of the pH indicator applied, so that it does not interfere with the ability of the pH indicator to change color in the presence of bacterial growth. In some embodiments, the adhesive is a medical grade adhesive when the polymeric indicator is for medical application. In some embodiments, the adhesive is an adhesive that meet all standards, as set forth by the FDA for food contact and/or food additives.

In some embodiments, the hydrophilic, hydronium ion penetrating uncolored transparent layer comprises a polymer selected from the group consisting of polyether block amide (e.g., Pebax®), (poly)hydroxyethyl methacrylate, (poly)hydroxypropyl methacrylate, (poly)glycerol methacrylate, copolymers of hydroxyethyl methacrylate, hydroxypropyl methacrylate or glycerol methacrylate and methacrylate acid, aminoacrylate and aminomethacrylate, (poly)vinyl pyrrolidone, (poly)vinylpyridine, polar polyamides, methyl cellulose, hydroxypropyl cellulose, hydroxyethyl cellulose, hydroxypropyl methylcellulose, ethyl hydroxyethylcellulose, carboxymethyl cellulose, cellulose acetate, cellulose acetate butyrate, cellulose acetate propionate, cellulose nitrate, polyvinyl acetate, polyvinyl alcohol, copolymers of polyvinyl acetate and polyvinyl alcohol, hydroxyl modified copolymers of vinyl acetate and vinyl chloride, polyesters, polyurethanes containing at least about 10% by weight of polyethylene oxide, styrene/methacrylic acid/hydroxyethyl methacrylate copolymers, styrene/methacrylic acid/hydroxypropyl methacrylate copolymers, methylmethacrylate/methacrylic acid copolymers, ethyl methacrylate/styrene/methacrylic acid copolymers, ethyl methacrylate/methyl methacrylate/styrene/methacrylic acid copolymers, polytetrafluoroethylene and hydrophilic cellulose copolymers.

In some embodiments, the hydrophobic, water impermeable layer comprises a polymer selected from the group consisting of polyethylene, polyethylene terephthalate, poly(vinylidene fluoride), poly(vinyl chloride), poly(vinylidene chloride), poly(vinyl alcohol), polypropylene, polyethylene, phenoxy resins, butadiene/styrene copolymers, butadiene/methylstyrene copolymers, poly(meth)acrylates, butadiene/acrylonitrile copolymers, ethylene/propylene copolymers, polybutadiene, polyisoprene, poly(oxy-2,6-dimethyl-1,4-phenylene), poly(oxycarbonyloxy-1,4-phenyleneisopropylidene-1,4-phenylene), acrylonitrile styrene copolymers, acrylonitrile/methyl acrylate/butadiene copolymers, acrylonitrile/styrene/butadiene copolymers, poly-1-vinylnaphthalene, polyvinylphenyl ketone, poly-p-xylenedodecanedioate, poly-tetramethylene octenediamide, poly-tetramethylene terephthalene, poly-trimethylene-3,3′-dibenzoate, poly-terephthallic anhydride, poly-4-methyl-diamine, polyvinylene carbonate, polyvinylene laurate, polyisopropenyl acetate, polyallylbenzene, polyvinylbutyl ether, polyvinyl formate, polyvinyl phenyl ether, polynorbornadine, polycarbonate, hydrophobic polyesters and polyurethanes, and many other resins, or a mixture thereof.

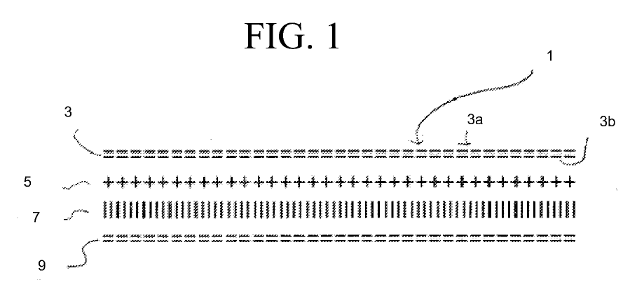

As shown in the attached FIG. 1, one example of a polymer film prepared by the processes described herein is a sandwich film. In particular, polymeric film 1 comprises an outer barrier layer polymer 3 which is a hydrophobic, water impermeable polymer as described below. Polymer layer 3 comprises a first and second surface, 3a and 3b respectively. In one preferred embodiment, an adhesive layer 5 is applied to the second surface 3b of outer barrier layer polymer 3. Application of the adhesive layer 5 can be conducted in any manner known in the art and the specific means for applying such a layer is not part of the invention. In one embodiment, the adhesive layer 5 can be applied by spraying an adhesive solvent system onto the second surface 3b. In another embodiment, the adhesive layer 5 can be applied by solvent casting wherein the solution of the adhesive and the solvent are applied to surface 3b (facing upward) and a conventional spreader is used to spread a uniform thickness of the solution to that surface followed by partial or complete drying of the solvent. Indicator layer 7 is applied in a manner likewise to that of adhesive layer 5 albeit each layer may be applied in the same or different manner. For example, indicator layer 7 can be applied by solvent casting and adhesive layer 5 can be applied by spraying. The inner or inside barrier layer 9 is then attached to the indicator layer in such a manner that a cohesive laminated polymer film is formed. “Cohesive” as used herein means that visually, the laminated polymeric layered film will not readily separate and is viewed as a single film.

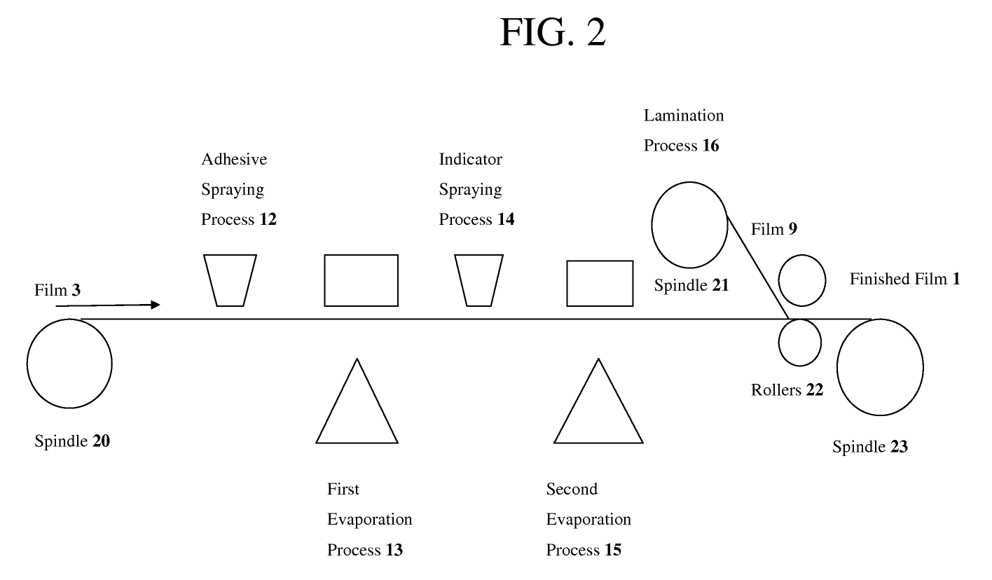

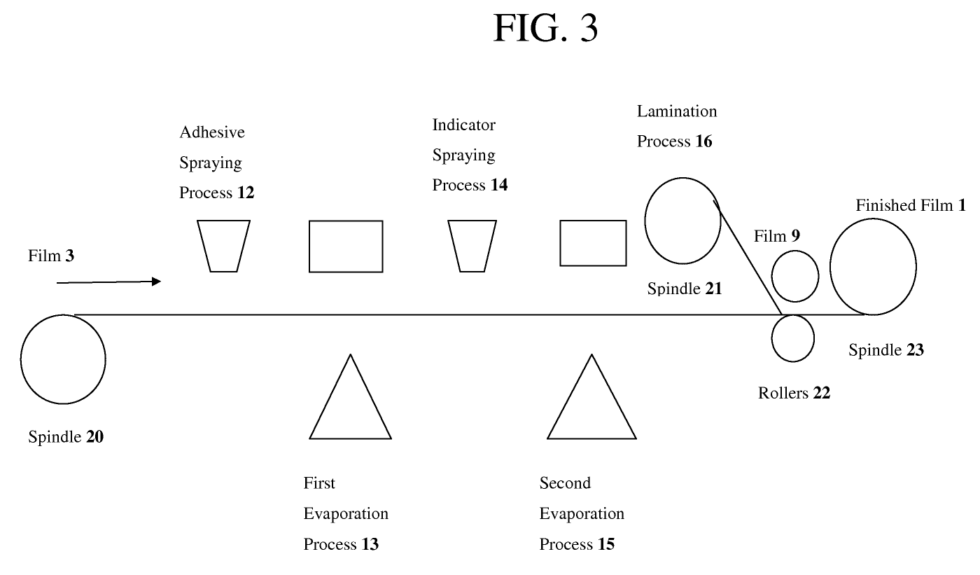

In some embodiments the polymeric indicator film is for application as a food wrap. In some embodiments of the food wrap application, the width of the source roll and the finished film roll can be approximately twelve (12) inches to up to about 84 inches. In some embodiments, the finished polymeric indicator film can be collected and wound around the collection roll with the top side or interior side to the outside of the collection roll as seen in FIG. 2. In some embodiments, the finished product roll of film can be collected on and wound around the collection roll with the top side or interior side to the inside of the collection roll as seen in FIG. 3.

In some embodiments, a release liner may be introduced as a second laminate process just prior to the line being wound onto the finished film roll. In some embodiments, a release liner can be employed for added separation of the film as it is wound onto the collection roll.

### Polymeric Indicator Films

In another aspect, this invention provides a polymeric indicator film comprising:
a) an inner layer comprising one or more hydrophilic, hydronium ion penetrating layers;
b) an outer layer comprising one or more hydrophobic, water impermeable layers; and
c) a pH indicator layer placed between the inner and outer layer.

In some embodiments, the pH indicator layer has a thickness of from 200 Angstroms to 5 microns.

In some embodiments, the polymeric indicator film further comprises an adhesive layer placed between the outer hydrophobic, water impermeable layer and the pH indicator layer.

### Food Spoilage Adaptation

In some embodiments, the polymeric indicator films produced by the process of this invention are films used on food or in bags into which foods are placed to detect the presence of metabolic byproducts from microorganisms. The pH change can be caused by numerous sources, including: gases, liquids containing electrolytes, ions and molecules that influence pH like lactic acid, citric acid and ammonia. As the definition of pH is the negative log of the hydrogen ion concentration, used to express the acidity or alkalinity of a solution, moieties which effect this ionic concentration change may be detectable.

The food wrap application is intended to be consistent with most films used to store low acid foods or left-overs such as Saran™ or Glad® wrap products or the film can be employed in the presentation of low acid foods at the point of sale. Low acid foods include meats, poultry, dairy, seafood and the like. These low acid foods have an inherent pH of near neutral or pH 7 or between pH 7.4 and 6.2. Foods known to be within the class referred to as medium acid foods are soups and pasta and have an inherent pH of 4.5 to 5.0. Foods that are known to be within the class referred to as acid foods are fruits and vegetables with an inherent pH between 3.7 and 4.5. Use of this product with foods other than low acid foods may result in a false-positive reaction of the indicator film.

In some embodiments of the food wrap application, the hydrophobic, water impermeable outer layer of the polymeric indicator film is resistant to the passage of environmental and ambient gaseous compounds such as oxygen, hydrogen, nitrogen, moisture or other elements.

### Medical Device Adaptation

In some embodiments, the polymeric indicator films produced by the process of this invention are films used for visually detecting bacterial growth in a medical setting, for example, related to wound dressings or catheter insertion sites. The presence of metabolic byproducts from microorganisms is detected by a change in the color of the polymeric indicator film positioned at or proximal to the site of wounds or catheter insertion sites.

In some embodiments, the polymeric indicator film for medical application that is applied to a patient's skin as a wound dressing uses hexamethoxy red as the indicator, which has a lower pKa than does heptamethoxy red.

In some embodiments of the polymeric indicator film for medical application, the outer layer has porosity such that it is resistant to the passage of a dipole molecule such as water but is permeable by environmental and ambient gaseous compounds such as oxygen, hydrogen, and nitrogen. In some embodiments, the outer layer comprises a polymer selected from the group consisting of acrylonitrile styrene copolymers, acrylonitrile/methyl acrylate/butadiene copolymers, acrylonitrile/styrene/butadiene copolymers, butadiene/acrylonitrile copolymers, butadiene/methylstyrene copolymers, butadiene/styrene copolymers, ethylene/propylene copolymers, polymethyl methacrylates, phenoxy resins, poly(meth)acrylates, poly(oxy-2,6-dimethyl-1,4-phenylene), poly(oxycarbonyloxy-1,4[1,4-phenyleneisopropylidene-1,4-phenylene), poly-1-vinylnaphthalenes, poly-4-methyl-diamines, polyallylbenzenes, polybutadienes, polyethylenes, polyisoprenes, polyisopropenyl acetates, poly-p-xylenedodecanedioate, poly-terephthallic anhydrides, poly-tetramethylene octenediamides, poly-tetramethylene terephthalenes, poly-trimethylene-3,3′-dibenzoates, polycarbonates, polyesters, polynorbornadines, polyurethanes, polyvinyl chlorides, polyvinyl fluorides, polyvinyl formates, polyvinylbutyl ethers, polyvinyl phenyl ether, polyvinylene carbonates, polyvinylene laurates, polyvinylidene chlorides, polyvinylidene fluorides, polyvinylphenyl ketones, polyether block amide (e.g., Pebax®), or a mixture thereof.

### EXAMPLES

### Example 1

### Preparation of a Polymeric Indicator Film as a Food Wrap

As shown in the FIG. 2 the food warp manufacturing process begins with a hydrophobic, water impermeable film 3, for example a polyethylene film such as Glad® wrap, which is the outer layer of the polymeric indicator film. The characteristic of the hydrophobic, water impermeable film 3 is its resistance to the passage of environmental and ambient gaseous compounds such as oxygen, hydrogen, nitrogen, moisture or other elements. The film 3 is brought to an adhesive spraying process 12, for example by the movement of the first spindle 20, where it is sprayed with an adhesive, preferably in a light spray or mist form. The adhesive may comprise one or more compounds in an aerosol. The film is then moved through the adhesive spraying process 12 to a first evaporation process 13 where an initial drying process of the adhesive begins. The adhesive may have a drying time of about five minutes at ambient temperatures and normal humidity conditions. The drying process may be accelerated by the addition of heat and/or forced airflow. The amount of drying can be controlled by the speed of the film through the first evaporation process 13, the temperature of the heat applied and/or the amount of forced air moved across the surface. The objective is to initiate evaporation of the carrier of the adhesive, such as acetone, while allowing the residual adhesive to accept the indictor, for example, a diluted heptamethoxy red used for the food warp application. The heat and/or forced air applied should be limited so as not to adversely affect the outer layer and/or the adhesive. A fume hood can be provided to collect the carrier vapors in a safe manner and dispose of the same in accordance with environmental regulatory requirements.

After the film has passed through the first evaporation process 13, it goes to the indicator spraying process 14 in which the adhesive receives an application of the indicator, for example, a dilute heptamethoxy red in a carrier, such as heptamethoxy red in 95% ethanol. The indicator is preferably applied in a light spray or mist form. The indicator heptamethoxy red in ethanol is applied to the surface of the drying adhesive in such a manner as to have the indicator immobilized on the surface of the drying adhesive. The film then passes though a second evaporation process 15 for evaporation of the carrier of the indicator. Heat and/or forced air may be applied to assist in the evaporation. The heat and/or forced air applied should be limited so as not to adversely affect the outer layer, the adhesive and/or the indicator. Once again a fume hood may be provided to collect the carrier vapors in a safe manner and dispose of the same in accordance with environmental regulatory requirements. Both the adhesive and the indicator are in the drying condition as the process moves into the lamination process 16. In the lamination process 16, a hydrophilic, hydronium ion penetrating film 9, which is the inner layer of the polymeric indicator film and may be supplied by the second spindle 21, is applied in a conventional method which carefully and gently presses the two layers together, for example by operation of rollers 22. The hydrophilic, hydronium ion penetrating film 9 may be a film with a relative high Moisture Vapor Transmission Rate (MVTR) such as Pebax® MV 3000 film. The residual adhesive should be sufficient to provide added laminate qualities to the film and contain the assembly as a “sandwich” containing the adhesive and immobilized indicator between the inner and outer layers. A second laminate process using a release liner, which is not shown in FIG. 2, can be applied to the film(s) just prior to collection on a finished polymeric indicator film 1 by the third spindle 23.

### Example 2

### Preparation of a Polymeric Indicator Film for Medical Application

As shown in FIG. 3 the manufacturing process of a polymeric indicator film for medical application begins with hydrophobic, water impermeable film 3. Preferably the hydrophobic, water impermeable film 3 used in a medical application is characterized by its passage of environmental and ambient gaseous compounds, such as oxygen, hydrogen, and nitrogen, through the “barrier”. The porosity of film 3 is such that it is resistant to the passage of a dipole molecule such as water. For example, film 3 may be a polyurethane film similar to that used by 3M™ in its Tegaderm™ product. Film 3 is brought to an adhesive spraying process 12, for example by the movement of the first spindle 20, where it is first sprayed with an adhesive, preferably in a light mist or spray form. The adhesive may comprise one or more compounds in an aerosol. The film then is moved through the adhesive spraying process 12 and on to a first evaporation process 13 where an initial drying process of the adhesive begins. The adhesive may have a drying time of about five minutes at ambient temperatures and normal humidity conditions. The drying process may be accelerated by the addition of heat and/or forced airflow. The amount of drying can be controlled by the speed of the film through the first evaporation process 13, the temperature of the heat applied and/or the amount of forced air moved across the surface. The objective is to initiate evaporation of the carrier while allowing the residual adhesive to accept the indictor, for example, a diluted hexamethoxy red used for the medical application. The heat applied should be limited so as not to adversely affect the outer layer and/or the adhesive. A fume hood can be provided to collect the carrier vapors in a safe manner and dispose of the same in accordance with environmental regulatory requirements.

After the film has passed through the first evaporation process 13, it goes to the indicator spraying process 14 in which the adhesive receives an application of the indicator, for example, a dilute hexamethoxy red in a carrier, such as ethanol. The indicator is preferably applied in a light spray or mist form. The indicator hexamethoxy red spray in ethanol is applied to the surface of the drying adhesive in such a manner as to have the indicator immobilized on the surface of the drying adhesive. The film then passes though a second evaporation process 15 to assist in the evaporation of the carrier of the indicator. Heat and/or forced air may be applied to assist in the evaporation. The heat and/or forced air applied should be limited so as not to adversely affect the outer layer, the adhesive and/or the indicator. Once again a fume hood may be provided to collect the carrier vapors in a safe manner and dispose of same in accordance with environmental regulatory requirements. Both the adhesive and the indicator are in the drying condition as the process moves into the lamination process 16. During the lamination process 16, hydrophilic, hydronium ion penetrating film 9, which is the inner layer of the polymeric indicator film and may be supplied by the second spindle 21, is applied in a conventional method which carefully and gently presses the two films together, for example by operation of the rollers 22. The film 9 may be a film with a relative high Moisture Vapor Transmission Rate (MVTR) such as Pebax® MV 3000 film or a polyurethane film with a high MVTR. The residual adhesive should be sufficient to provide added laminate qualities to the film and contain the assembly as a “sandwich” containing the adhesive and immobilized indicator between the inner and outer layers. A second laminate process using a release liner, which is not shown in FIG. 3, can be applied to the film(s) just prior to collection on finished polymeric indicator film 1 by the third spindle 23.

Additional rollers, which are not shown in FIGS. 2 and 3 may be used to support the film from above and/or below as it moves through the sequenced series of applications. These rollers can be employed not only to support the film from above and/or below but to mitigate any wrinkling of the film during the manufacturing processes.

### Example 3

### Application of a Polymeric Indicator Film on Beef

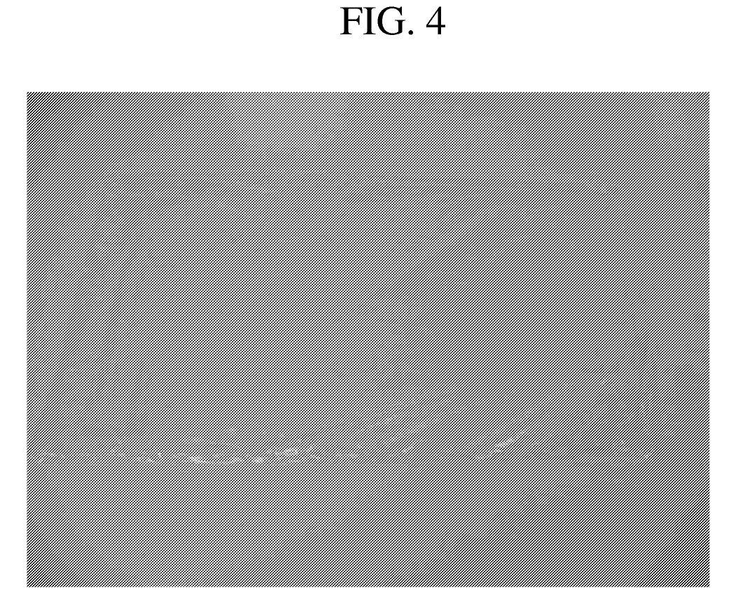

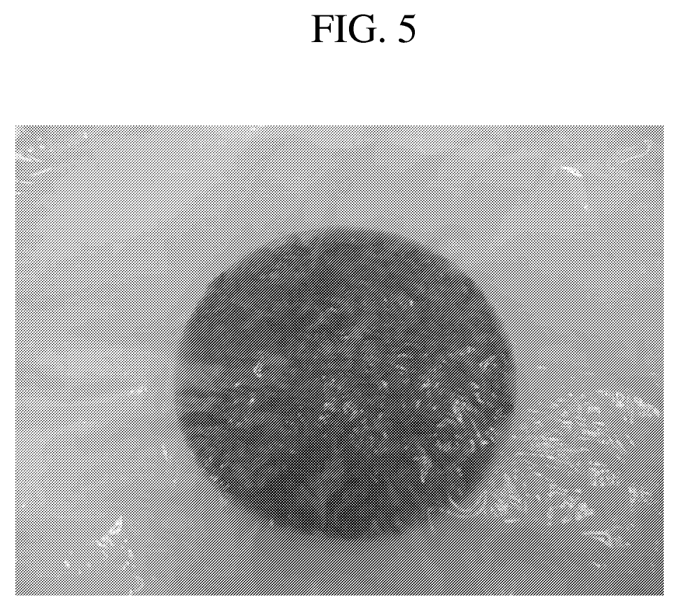

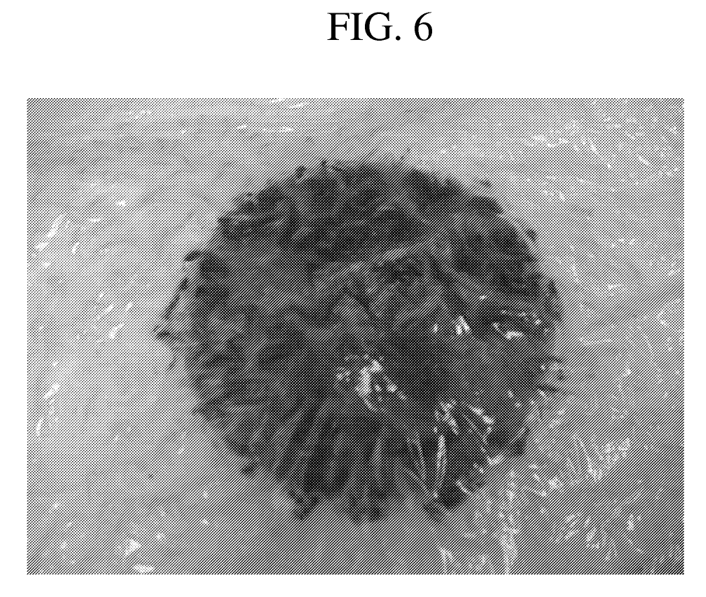

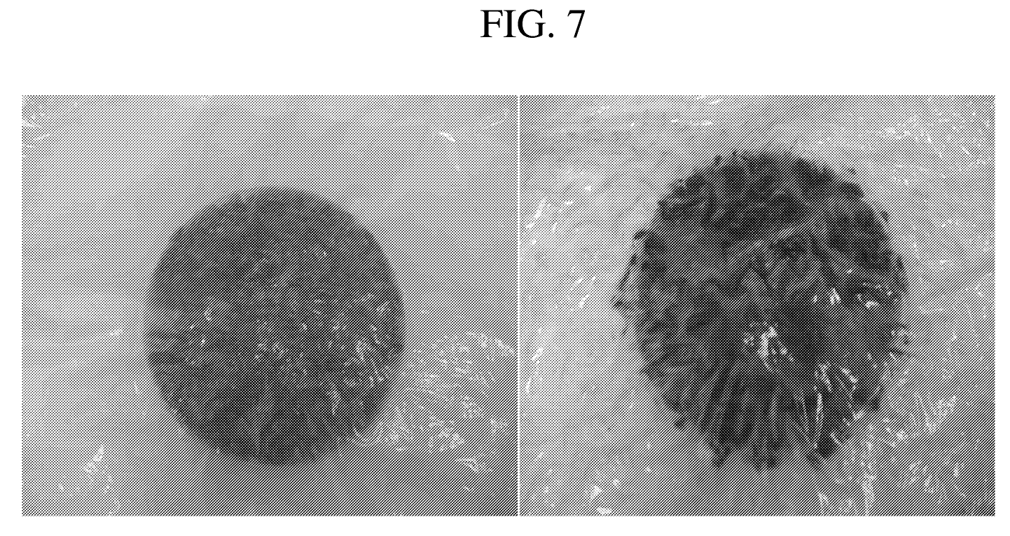

FIG. 4 illustrates a polymeric indicator film prior to its use on a food product. It was applied to a sample of ground beef contaminated with Escherichia coli (E. coli) of less than 100 colony forming units (CFUs) at ambient conditions (FIG. 5). FIG. 6 which was taken after 18 hours shows the red color appearing on the original uncolored polymeric indicator film indicating the presence of bacteria. FIG. 7 is a side-by-side comparison of the polymeric indicator film before and after detection of the bacteria presence.

### Example 4

### Application of a Polymeric Indicator Film on Chicken

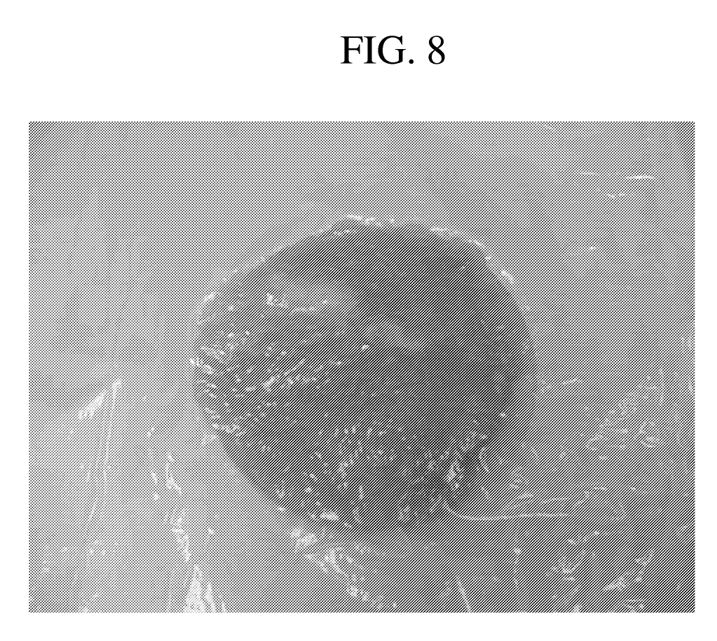

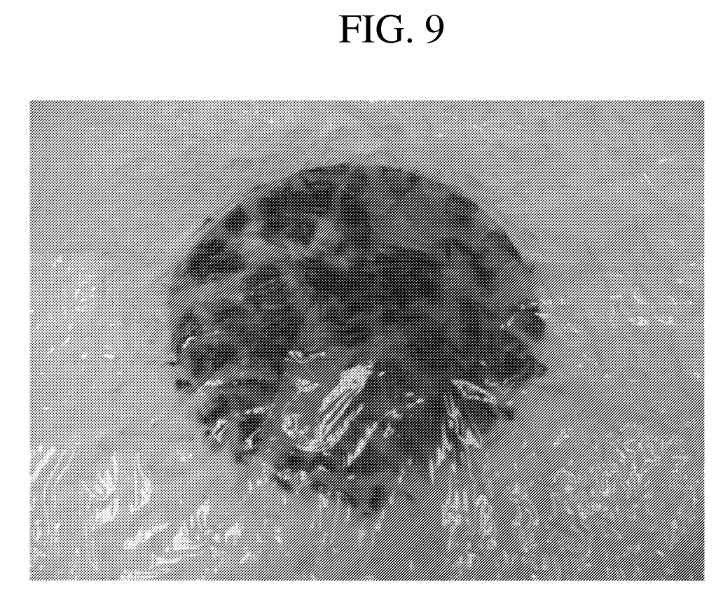

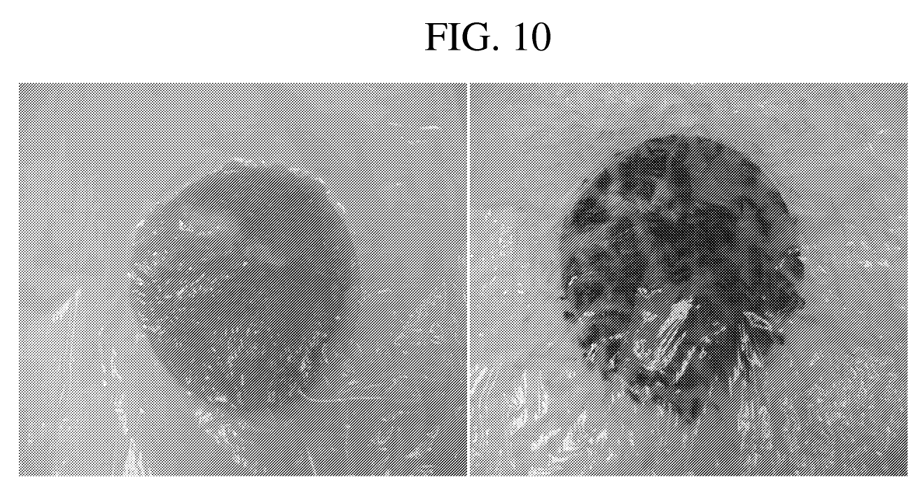

A polymeric indicator film was applied to a sample of chicken contaminated with Salmonella of less than 100 CFUs at ambient conditions (FIG. 8). FIG. 9 which was taken after 18 hours shows the red color appearing on the original uncolored polymeric indicator film indicating the presence of bacteria. FIG. 10 is a side-by-side comparison of the polymeric indicator film before and after detection of the bacteria presence.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A process for preparing a polymeric indicator film comprising an inner hydrophilic, hydronium ion penetrating transparent layer and an outer hydrophobic, water impermeable transparent layer which process comprises:
a) selecting one or more hydrophilic, hydronium ion penetrating layers as the inner layer of the polymeric film wherein the inner layer has a first and second surface;
b) selecting one or more hydrophobic, water impermeable layers as the outer layer of the polymeric film wherein the outer layer has a first and second surface;
c) applying a pH indicator layer to at least a portion of one surface of the outer layer; and
d) bonding the inner and outer surfaces together such that the pH indicator layer is placed between the inner and outer layers.

2. A process for preparing a polymeric indicator film comprising an inner hydrophilic, hydronium ion penetrating transparent layer and an outer hydrophobic, water impermeable transparent layer which process comprises:
a) selecting one or more hydrophilic, hydronium ion penetrating layers as the inner layer of the polymeric film wherein the inner layer has a first and second surface;
b) selecting one or more hydrophobic, water impermeable layers as the outer layer of the polymeric film wherein the outer layer has a first and second surface;
c) applying an adhesive layer to one surface of the outer layer;
d) applying a coating of pH indicator to at least a portion of the adhesive layer; and
e) bonding the inner and outer surfaces together such that the adhesive layer coated with the pH indicator is placed between the inner and outer layers.

3. The process of claim 2, wherein the adhesive is partially dried before the coating of pH indicator is applied.

4. The process of claim 2, wherein the outer layer with the indicator applied thereto is dried before binding of the outer layer with the inner layer.

5. The process of claim 1, wherein the pH indicator is selected from the group consisting of xylenol blue (p-xylenolsulfonephthalein), bromocresol purple (5′,5″-dibromo-o-cresolsulfonephthalein), bromocresol green (tetrabromo-m-cresolsulfonephthalein), cresol red (o-cresolsulfonephthalein), phenolphthalein, bromothymol blue (3′,3″-dibromothymolsulfonephthalein), p-naphtholbenzein (4-[alpha-(4-hydroxy-1-naphthyl)benzylidene]-1(4H)-naphthalenone), neutral red (3-amino-7-dimethylamino-2-methylphenazine chloride), hexamethoxy red and heptamethoxy red, and combinations thereof.

6. The process of claim 1, wherein the pH indicator comprises hexamethoxy red.

7. The process of claim 1, wherein the pH indicator comprises heptamethoxy red.

8. The process of claim 1, wherein the polymeric indicator film is used as a food wrap.

9. The process of claim 1, wherein the polymeric indicator film is used in a medical setting.

10. A polymeric indicator film comprising:
a) an inner layer comprising one or more hydrophilic, hydronium ion penetrating layers;
b) an outer layer comprising one or more hydrophobic, water impermeable layers;
c) a pH indicator layer placed between the inner and outer layer.

11. The polymeric indicator film as defined in claim 10 wherein the pH indicator layer has a thickness of from 200 Angstroms to 5 microns.

12. The polymeric indicator film as defined in claim 10 further comprises an adhesive layer placed between the outer hydrophobic, water impermeable layer and the pH indicator layer.
